(19) United States
(12) Patent Application Publication
(10) Pub. No.: US 2012/0172748 A1
(43) Pub. Date: Jul. 5, 2012

(54) Temperature measuring device

(76) Inventor: Lisa A. Dunn, Kihei, HI (US)

(21) Appl. No.: 12/930,129

(22) Filed: Dec. 29, 2010

(51) Int. Cl. A61B 5/01

(52) U.S. Cl. 600/549

(57) ABSTRACT

An apparatus for accurately measuring the body temperature of a patient at the tracheal wall of the patient. The apparatus includes a novel handheld temperature readout assembly that is easy to use by the caregiver and one which clearly and accurately displays the temperature of the patient. The handheld temperature readout assembly includes a handheld housing having a viewing window and a light emitting diode temperature display that is carried by the housing and is readily viewable through the viewing window of the handheld housing for displaying the patient temperature. The apparatus also includes a built-in alarm system to alert to high and low body temperatures.

### CROSS-REFERENCE TO RELATED APPLICATIONS

Not Applicable

### STATEMENT REGARDING FEDERALLY SPONSORED RESEARCH OR DEVELOPMENT

Not Applicable

### INCORPORATION-BY-REFERENCE OF MATERIAL SUBMITTED ON A COMPACT DISC

Not Applicable

### BACKGROUND OF THE INVENTION

1. Field of the Invention

The present invention relates generally to apparatus for measuring body temperature. More particularly, the invention concerns an apparatus for measuring tracheal temperatures.

2. Description of Related Art Including Information Disclosed Under 37 CFR 1.97 and 1.98

A number of different types of temperature measuring apparatus using various types of temperature sensing devices such as thermisters, thermocouples and resistance temperature sensors have been suggested in the past. Typically, the prior art temperature sensing devices are placed under the arm, in the ear or in the rectum. Additionally, temperature is sometimes measured by temporal scanning techniques.

Special problems exist in measuring and monitoring the temperature of neonatal and pediatric patients. For example, neonatal intensive care units typically confine newborns in an incubator until their temperature stabilizes and their body weight reaches a desired number. As a general rule, their temperature is monitored by a probe connected by way of an adhesive-like patch to their body and to an electrical monitor that has a digital readout. Once the temperatures and body weight of the neonate reaches an acceptable number, the neonate is placed in an open bassinet (baby crib). When in the bassinet, the neonate's temperature is typically monitored by the nurse using a disposable battery powered digital thermometer that is placed under the baby's axilla (armpit). During this process, the baby's arm has to remain still and close to the body in order to obtain accurate temperature readings. Due to their medical condition, many neonates are irritable and have a hyper-active (Moro) startle reflex, making it difficult for them to hold still and thereby causing inaccurate temperature readings. Additionally, having a thermometer held under the armpit further upsets the neonate, resulting in crying which often causes an abnormal temperature result. Since much of the care that is given to the neonate is based on temperature findings, inaccurate temperature readings can adversely affect the neonate's care and treatment outcome.

By way of example, doctors' orders based on temperature findings would read as follows:
1. Blood cultures ×2 for all temperatures above 102° F. (Fahrenheit).
2. Acetaminophen (per body weight) every 4 hours, if needed, for temperatures >(greater) than 102° F. (Fahrenheit) after blood cultures.

With the foregoing in mind, an inaccurate temperature reading would prevent:
1. The proper care being implemented (for example, no lab work ordered to determine the organism).
2. The starting of an antibiotic earlier in treatment, preventing possible sepsis (organism traveling via the blood stream and causing potential organ damage, failure, and possible death).
3. Early fever detection and treatment to prevent febrile seizures [seizures caused by elevated body temperatures (fevers)] and possible brain damage as a result of hypoxia from the seizure, causing deprivation of oxygen to the brain.

It is the foregoing problems that the apparatus of the present invention seeks to at least partially address.

When the patient, and particularly a neonatal or pediatric patient, is to be fitted with a tracheal tube, a unique opportunity is presented to use the apparatus of the present invention to accurately measure the patient's body temperature in the trachea.

By way of brief background, a tracheal tube is a catheter that is inserted into the trachea for the purpose of establishing and maintaining a patient airway and to ensure the adequate exchange of oxygen and carbon dioxide. Many different types of tracheal tubes are available. For example, an endotracheal tube is a specific type of tracheal tube that is nearly always inserted through the mouth (orotracheal) or nose (nasotracheal). Another common type of tracheal tube is a tracheostomy tube, which is a curved tube that is inserted into a tracheostomy stoma (the hole made in the neck and windpipe).

By being able to check the patient's body temperature from a temperature measuring device as part of their ET (endotracheal) tubes and trach (tracheostomy) tube, the doctor would have accurate data to implement patient care by and more frequent monitoring for critical patients, those with fragile medical conditions, and those that need more frequent monitoring. This would allow the paramedics to provide a quick and accurate assessment at the scene for the patient's care and to relay the information to the doctor.

Additionally, an alarm on the ET tube or trach tube functions to alert the caregiver that there is a temperature elevation (fever) and provides prompt notice of a spike in body temperature that might otherwise be delayed in detecting.

Having an alarm to alert that there is a lower than therapeutic body temperature (hypothermia), especially in newborns, can be life saving. Hyperthermia can occur rapidly and without warning.

### BRIEF SUMMARY OF THE INVENTION

By way of brief summary, the apparatus of the present invention is specially designed for use with:
(a) intubated patients having an endotracheal tube manually inserted via the nares (nasal opening) and via the mouth into the trachea for artificial ventilation of the lungs during cardio pulmonary resuscitation, drowning, electrocutions, traumas and similar life-threatening situations;
(b) patients with a trach (tracheostomy) tube inserted surgically through the neck into the trachea, held in place by a trach tie (belt-like) device in hospital units and/or at home.

With the foregoing in mind, the apparatus of the invention can be used in emergency rooms, in all levels of pediatric and neonatal (newborn) intensive care units, in adult intensive care units including, but not limited to cardiac, surgical, and medical, in all levels of care and in any unit of the hospital where frequent and close monitoring of a patient's temperature is vital to their care and will produce a better outcome for the patient.

Additionally, the apparatus of the invention can effectively be used for patients on ECMO (extra corporeal membrane oxygenation) that require close monitoring and in situations where the patient exhibits respiratory distress, weak or diseased heart or lungs that require artificial means of removing carbon monoxide from their lungs and exchanging it for oxygenated blood through a mechanical filter hooked up via large veins by an intravenous catheter (tube). In this latter instance, it has long been recognized that these vein puncture access sites are a source of infection and lead to sepsis resulting in up to 25% fatalities. By having a less time consuming and more accurate way to monitor the temperatures of these patients, infections could be detected earlier and patient outcomes would be improved.

In light of the foregoing, it is an object of the present invention to provide an apparatus for accurately measuring the body temperature of a patient at the tracheal wall of the patient.

Another object of the invention is to provide an apparatus of the aforementioned character that is self-contained and does not have to be interconnected with expensive monitoring equipment.

Another object of the invention is to provide an apparatus of the character described that can be used in any medical setting as well as in the home care environment and one that can be used by a relatively untrained caregiver.

Another object of the invention is to provide an apparatus as described in the preceding paragraphs that includes a built-in alarm system to alert to high and low body temperatures.

Another object of the invention is to provide an apparatus of the class described that includes a novel handheld temperature readout assembly that is easy to use by the caregiver and one which clearly and accurately displays the temperature of the patient. More particularly, it is an object of the invention to provide such an apparatus in which the handheld temperature readout assembly includes a handheld housing having a viewing window and a light emitting diode temperature display that is carried by the housing and is readily viewable through the viewing window of the handheld housing for displaying the patient temperature.

Another object of the invention is to provide an apparatus of the character described that includes an inflatable cuff that is disposed about the interior end of the tracheal tube.

The foregoing as well as other objects of the invention will be achieved by the apparatus illustrated in the accompanying drawings and described in the specification which follows.

### BRIEF DESCRIPTION OF SEVERAL VIEWS OF THE DRAWINGS

FIG. 1 is a generally perspective view of one form of the temperature measuring apparatus of the invention.

FIG. 2 is a generally perspective view of an alternate form of the temperature measuring apparatus of the invention.

FIG. 3 is a generally perspective view of still another form of the temperature measuring apparatus of the invention.

FIG. 4 is a generally perspective view of yet another form of the temperature measuring apparatus of the invention.

FIG. 5 is an enlarged, generally perspective exploded view of one form of the handheld temperature readout module of the apparatus of the invention.

### DETAILED DESCRIPTION OF THE INVENTION

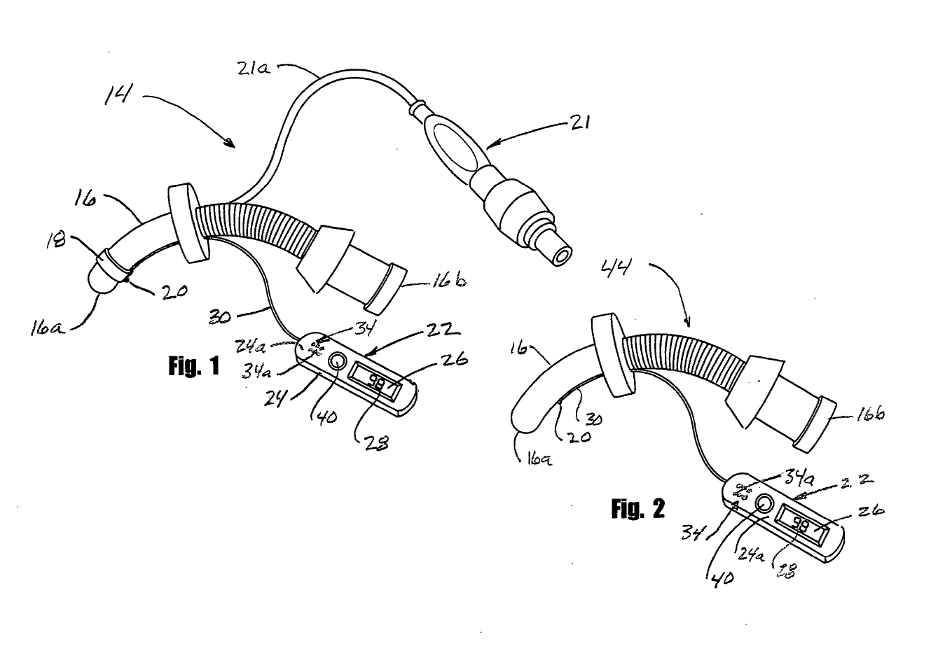

Referring to the drawings and particularly to FIGS. 1 and 5, one form of the temperature measuring apparatus of the invention for measuring the temperature of the patient is there illustrated and generally designated by the numeral 14. This form of the apparatus here comprises a tracheostomy tube 16 that has a curved interior end portion 16a and an exterior end 16b. The interior end 16a of the tube is constructed and arranged to enter the trachea of the patient via a tracheostomy stoma in a conventional manner well understood by those skilled in the art. Provided on the interior end portion 16a of the tube is a conventional, inflatable cuff 18 that is so constructed and arranged as to engage the wall of the trachea of the patient when inflated. As illustrated in FIG. 1 of the drawings, a temperature sensor 20 in the form of a temperature-responsive element is connected to the inflatable cuff 18 and is arranged to engage the trachea wall of the patient when the tube is in position within the trachea of the patient and the cuff is inflated. While the temperature sensor or temperature responsive element 20 can take various forms well known by those skilled in the art, the temperature sensor here comprises a conventional thermistor. Interconnected with inflatable cuff 18 by means of an elongated fill tube 21a is a conventional hand operated pump assembly 21.

An important feature of the apparatus of the present invention is a handheld temperature readout assembly 22 that is operably interconnected with the temperature sensor 20 and functions to clearly and accurately display the temperature of the patient.

In the present form of the invention, the novel temperature readout assembly 22 comprises a handheld housing 24 having a viewing window 26 and a light emitting diode temperature display 28 that is carried by the housing and is clearly viewable through the viewing window 26. Temperature display 28 displays the temperature detected by the temperature sensor 20, which in a manner well understood by those skilled in the art, converts ambient temperature into an electronic signal. As illustrated in FIGS. 1 and 5 of the drawings, an electrical connector 30 interconnects the temperature sensor 20 with a printed circuit board 32 that includes an electronic processor of conventional design, which is operably associated with the light emitting diode temperature display 28. In a manner well understood by those skilled in the art, the electronic processor of the printed circuit board 32 converts the electronic signals from temperature sensor 20 into digital signals that are displayed by temperature display 28. Printed circuit board 32 is also operably interconnected with a built-in alarm system 34 of conventional construction that is housed within housing 24 and functions to alert the caregiver to high and low patient body temperatures detected by the temperature sensor 20. Alarm system 34 includes a speaker array 34a that is mounted on the upper wall 24a of housing 24 and functions to alert the caregiver to high and low body temperature readings.

As illustrated in FIG. 5 of the drawings, housing 24 includes an upper portion 24b and a lower portion 24c that are removably connected to the upper portion so as to permit the caregiver to gain access to the power source, or battery 38 that powers the light emitting diode temperature display 28, the printed circuit board 32 and the built-in alarm system 34. A conventional pushbutton switch 40, which is carried by the upper portion 24b of the housing, operates a power switch 42 that is operably associated with the printed circuit board 32 and functions to control the electrical connection between the battery 38 and the printed circuit board. While the apparatus of the invention illustrated in FIG. 1 of the drawings is usable with various types of patients, the apparatus illustrated is particularly well-suited for use in connection with neonates that are confined within an incubator. In such a situation, the handheld temperature readout of the apparatus can be easily operated and read by the caregiver and has been proven to be vastly superior to the more complex temperature readout devices typically found in conventional neonatal incubators.

Turning next to FIG. 2 of the drawings, another form of the temperature measuring apparatus of the invention for measuring the temperature of the patient is there illustrated and generally designated by the numeral 44. This form of the apparatus is similar in most respects to the apparatus illustrated in FIG. 1 of the drawings and like numerals are used in FIG. 2 to identify like components. The primary difference between this latest form of the invention and the earlier described embodiment resides in the fact that the embodiment of FIG. 2 does not include an inflatable cuff and therefore does not include a pump apparatus for inflating the inflatable cuff.

As in the earlier form of the invention, this latest embodiment comprises a tracheostomy tube 16 that is substantially identical in construction and operation to that previously described. As before, the curved interior end portion 16a of the tube 16 is constructed and arranged to enter the trachea of the patient via a tracheostomy stoma in a conventional manner well understood by those skilled in the art. Disposed on the interior end portion 16a of the tube is a temperature sensor 20 that is substantially identical in construction and operation to the previously described temperature sensor and is arranged to engage the trachea wall of the patient when the tube is in position within the trachea of the patient.

Operably associated with the temperature sensor 20 is a handheld temperature readout assembly 22 that is also substantially identical in construction and operation to that previously described. Once again, the novel temperature readout assembly 22 of this latest form of the invention comprises a handheld housing 24 having a viewing window 26 and a light emitting diode temperature display 28 that is carried by the housing and is clearly viewable through the viewing window 26. Temperature display 28 displays the temperature detected by the temperature sensor 20 which, in a manner well understood by those skilled in the art, converts ambient temperature into an electronic signal. An electrical connector 30 interconnects the temperature sensor 20 with a printed circuit board 32 that is housed within housing 24.

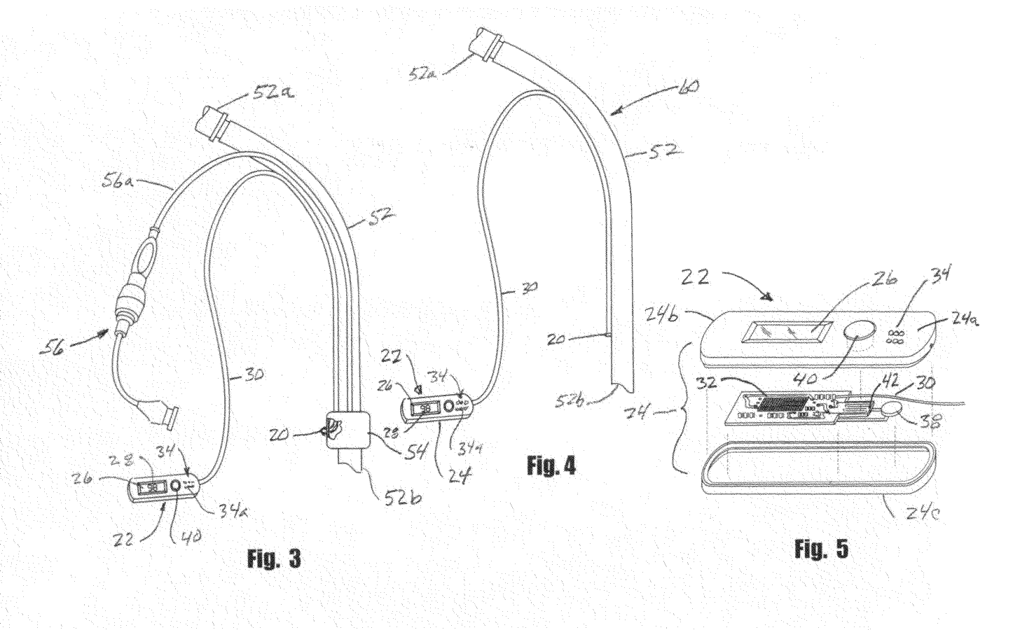

Referring now to FIG. 3 of the drawings, yet another form of the temperature measuring apparatus of the invention for measuring the temperature of the patient is there illustrated and generally designated by the numeral 50. This form of the apparatus is also similar in some respects to the apparatus illustrated in FIG. 1 of the drawings and like numerals are used in FIG. 3 to identify like components. The primary difference between this latest form of the invention and the earlier described embodiments resides in the fact that the embodiment of FIG. 3 comprises an endotracheal tube 52 rather than a tracheostomy tube. Endotracheal tube 52 has an exterior end portion 52a and an interior end portion 52b that are constructed and arranged to enter the trachea of the patient in a conventional manner via the mouth (orotracheal) or nose (nasotracheal).

Connected to the interior end portion 52b of the tube is a conventional, inflatable cuff 54 that is so constructed and arranged as to engage the wall of the trachea of the patient when inflated. As illustrated in FIG. 3 of the drawings, a temperature sensor 56 in the form of a temperature-responsive element is connected to the inflatable cuff 54 and is arranged to engage the trachea wall of the patient when the inner end portion 52b of the tube is in position within the trachea of the patient and the cuff is inflated. While the temperature sensor or temperature responsive element 56 can take various forms well known by those skilled in the art, the temperature sensor here comprises a conventional thermocouple. Interconnected with inflatable cuff 54 by means of an elongated fill tube 56a is a hand operated pump assembly 56 of a conventional construction.

As before, an important feature of the apparatus of this latest form of the invention is a handheld temperature readout assembly 22 that is operably interconnected with the temperature sensor 56 and functions to clearly and accurately display the temperature of the patient such as an infant. Temperature readout assembly 22, which is substantially identical in construction and operation to that previously described, comprises a handheld housing 24 having a viewing window 26 and a light emitting diode temperature display 28 that is carried by the housing. Temperature display 28 displays the temperature detected by the temperature sensor 56 which, in a manner well understood by those skilled in the art, converts ambient temperature into an electronic signal. As illustrated in FIGS. 3 and 5 of the drawings, an electrical connector 30 interconnects the temperature sensor 56 with a printed circuit board 32 that includes an electronic processor of conventional design which is operably associated with the light emitting diode temperature display 28. In a manner well understood by those skilled in the art, the electronic processor of the printed circuit board 32 converts the electronic signals from temperature sensor 56 into digital signals that are displayed by temperature display 28. Printed circuit board 32 is also operably interconnected with a built-in alarm system 34 of conventional construction that is housed within housing 24 and functions to alert the caregiver to high and low patient body temperatures detected by the temperature sensor 20. Alarm system 34 includes a speaker array 34a that is mounted on the upper wall 24a of housing and functions to alert the caregiver to high and low body temperature readings.

Turning finally to FIG. 4 of the drawings, yet another form of the temperature measuring apparatus of the invention is there illustrated and generally designated by the numeral 60. This form of the apparatus is similar in most respects to the apparatus illustrated in FIG. 3 of the drawings and like numerals are used in FIG. 4 to identify like components. The primary difference between this latest form of the invention and the embodiment of FIG. 3 resides in the fact that the embodiment of FIG. 4 does not include an inflatable cuff and therefore does not include a pump apparatus for inflating the inflatable cuff.

As in the earlier form of the invention, this latest embodiment comprises a tracheostomy tube 52 that is substantially identical in construction and operation to that previously described. As before, the interior end portion 52b of the tube 52 is constructed and arranged to enter the trachea of the patient via the mouth (orotracheal) or nose (nasotracheal) in a conventional manner well understood by those skilled in the art. Connected to the interior end portion 52b of the tube is a temperature sensor 20 that is arranged to engage the wall of the patient's trachea and is substantially identical in construction and operation to the previously described temperature sensor 20.

Operably associated with the temperature sensor 20 is a handheld temperature readout assembly 22 that is also substantially identical in construction and operation to that previously described. Once again, the novel temperature readout assembly 22 of this latest form of the invention comprises a handheld housing 24 having a viewing window 26 and a light emitting diode temperature display 28 that is carried by the housing and is clearly viewable through the viewing window 26. Temperature display 28 displays the temperature detected by the temperature sensor 20 which, in a manner well understood by those skilled in the art, converts ambient temperature into an electronic signal. An electrical connector 30 interconnects the temperature sensor 20 with a printed circuit board 32 that is housed within housing 24.

Once again, the apparatus illustrated in FIGS. 4 and 5 is particularly well-suited for use in connection with neonates. In such use, the handheld temperature readout of the apparatus can be easily operated read by the caregiver in the hospital and in the home and has been proven to be vastly superior to the more complex temperature readout devices typically found in neonatal temperature measuring devices.

Having now described the invention in detail in accordance with the requirements of the patent statutes, those skilled in this art will have no difficulty in making changes and modifications in the individual parts or their relative assembly in order to meet specific requirements or conditions. Such changes and modifications may be made without departing from the scope and spirit of the invention, as set forth in the following claims.

1. A temperature measuring apparatus for measuring the temperature of the patient comprising:
(a) a tracheal tube having an interior end and an exterior end, said interior end being constructed and arranged to enter the trachea of the patient and to engage the trachea wall;
(b) a temperature sensor interconnected with said tracheal tube proximate said interior end thereof, said temperature sensor being so constructed and arranged so as to engage the trachea wall of the patient;
(c) a handheld temperature readout assembly operably interconnected with said temperature sensor for displaying the temperature of the patient, said readout assembly comprising:
(i) a handheld housing having a viewing window; and
(ii) a light emitting diode temperature display carried by said housing and being viewable through said viewing window of said handheld housing for displaying the temperature detected by said temperature sensor.

2. The apparatus as defined in claim 1 in which said hand held temperature readout assembly further includes a switch operably associated with said temperature display for activating said temperature display.

3. The apparatus as defined in claim 1 in which said temperature sensor comprises a thermistor.

4. The apparatus as defined in claim 1 further including an inflatable cuff disposed proximate said interior end of said tracheal tube, said inflatable cuff being so constructed and arranged as to engage the wall of the trachea of the patient when inflated.

5. The apparatus as defined in claim 1 in which said tracheal tube comprises a tracheostomy tube.

6. The apparatus as defined in claim 1 in which said tracheal tube comprises an endotracheal tube.

7. A temperature measuring apparatus for measuring the temperature of an infant comprising:
(a) a tracheal tube having an interior end and an exterior end, said interior end being constructed and arranged to enter the trachea of the infant and to engage the trachea wall;
(b) a temperature sensor interconnected with said tracheal tube proximate said interior end thereof, said temperature sensor being so constructed and arranged so as to engage the trachea wall of the infant;
(c) a handheld temperature readout assembly operably interconnected with said temperature sensor for displaying the temperature of the infant, said readout assembly comprising:
(i) a handheld housing having a viewing window;
(ii) a light emitting diode temperature display carried by said housing and being viewable through said viewing window of said handheld housing for displaying the temperature detected by said temperature sensor; and
(d) a switch operably associated with said temperature display for activating said temperature display.

8. The apparatus as defined in claim 7 in which said temperature sensor comprises a thermistor.

9. The apparatus as defined in claim 7 in which said temperature sensor comprises a thermocouple.

10. The apparatus as defined in claim 7 in which said tracheal tube comprises a tracheostomy tube.

11. The apparatus as defined in claim 7 in which said tracheal tube comprises an endotracheal tube.

12. The apparatus as defined in claim 7 further including an inflatable cuff disposed proximate said interior end of said tracheal tube, said inflatable cuff being so constructed and arranged as to engage the wall of the trachea of the infant when inflated.

13. The apparatus as defined in claim 12 further including means carried by said tracheal tube for inflating said inflatable cuff.

14. A temperature measuring apparatus for measuring the temperature of a neonate comprising:
(a) a tracheostomy tube having a curved interior portion and an exterior portion connected to said curved interior portion, said interior portion being constructed and arranged to enter the trachea of the neonate and to engage the trachea wall of the neonate;
(b) a temperature sensor comprising a thermistor connected to said interior portion of said tracheostomy tube, said temperature sensor being so constructed and arranged so as to engage the trachea wall the neonate;
(c) a handheld temperature readout assembly operably associated with said temperature sensor for displaying the temperature of the neonate, said readout assembly comprising:
(i) a handheld hollow housing having an outer wall having a viewing window;
(ii) a light emitting diode temperature display carried by said housing and being viewable through said viewing window of said handheld housing for displaying the temperature detected by said temperature sensor; and
(iii) a switch operably associated with said light emitting diode of said temperature display for activating said light emitting diode.

15. The apparatus as defined in claim 14 further including an inflatable cuff connected to said interior portion of said tracheal tube, said inflatable cuff being so constructed and arranged as to engage the wall of the trachea of the neonate when inflated.
